(19) United States
(12) Patent Application Publication
(10) Pub. No.: US 2015/0182726 A1
(43) Pub. Date: Jul. 2, 2015

(54) Simultaneous Dual Catheter Control System And Method For Controlling An Imaging Catheter To Enable Treatment By Another Catheter

(71) Applicant: Catheter Robotics, Inc., Budd Lake, NJ (US)

(72) Inventor: David A. Jenkins, Flanders, NJ (US)

(73) Assignee: Catheter Robotics, Inc., Budd Lake, NJ (US)

(21) Appl. No.: 14/143,435

(22) Filed: Dec. 30, 2013

(51) Int. Cl. A61M 25/01; A61B 8/12; A61B 18/14; A61B 8/08

(52) U.S. Cl. CPC A61M 25/0108; A61B 8/0841; A61B 8/12; A61B 18/1492; A61B 8/52; A61M 25/0133; A61B 2018/00577

(57) ABSTRACT

Systems, devices, methods, and non-transitory processor-readable storage media for a computing device to generate repositioning instructions for a remotely-controlled catheter positioning system to position a first catheter co-located with a second catheter within a patient's organ, including performing image analysis on imagery generated based on data from the first catheter, calculating positioning corrections based on the performed image analysis, generating repositioning instructions based on the calculated positioning correction information, and transmitting control signals based on the generated repositioning instructions to the remotely-controlled catheter positioning device. The computing device may also determine whether a representation of the second catheter is within the imagery based on the performed image analysis and whether a representation of a tip of the second catheter is at a desired location within the imagery. In an embodiment, the first catheter may be an ultrasound catheter.

### BACKGROUND

Invasive procedures, such as ablation or electrophysiology procedures, often utilize catheters and similar devices to take measurements, receive imagery, and enable surgical functions. Such procedures are very complicated and often require additional techniques and devices to visualize the location of catheters within patients during the procedures. For example, a fluoroscopy imaging technique may be used to help position catheters or other elongated medical devices within a patient's body within an organ, such as the heart or the circulatory system. Because fluoroscopy imaging uses ionizing radiation, systems have been developed to enable the insertion and maneuvering of steerable catheters within patients using remote controls. These remote techniques may protect clinicians and attending nurses from exposure to high cumulative dosages of radiation, as well as provide fine control over catheters.

### SUMMARY OF THE INVENTION

The various embodiments include a system and methods for controlling a remotely controlled catheter positioning system to maintain a therapeutic or diagnostic catheter within the field of view of an ultrasound imaging catheter while both are positioned within a patient's body. The embodiments may enable improved medical procedures by relieving a clinician of a burden of frequently repositioning and imaging catheter while performing therapeutic or diagnostic procedures with another catheter.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIGS. 1A-1C are system block diagrams of catheter positioning systems that include at least a remote controller, a remotely-controlled catheter positioning device, and a computing device suitable for use with the various embodiments.

FIGS. 2A-2C are diagrams illustrating two catheters in various states of positioning within a patient as applicable to various embodiments.

FIGS. 3 and 4 are process flow diagrams illustrating embodiment methods for a computing device to direct a remotely-controlled catheter positioning system to position an ultrasound catheter to keep a second catheter within the field of view of the ultrasound image.

FIGS. 5A and 5B are process flow diagrams illustrating embodiment methods for a computing device to direct a first remotely-controlled catheter positioning system to position an ultrasound catheter to keep a second catheter within the field of view of the ultrasound image based on control signals provided to a second remotely-controlled catheter positioning system positioning the second catheter.

FIG. 6 is a component diagram of an ultrasound catheter suitable for use in various embodiments.

FIG. 7 is a component diagram of a catheter suitable for use in various embodiments.

FIG. 8 is a component diagram of components used by a remotely-controlled catheter positioning system suitable for use in various embodiments.

FIG. 9 is a component diagram of a remotely-controlled catheter positioning system suitable for use in various embodiments.

FIG. 10 is a component diagram of a computing device suitable for use in various embodiments.

FIG. 11 is a component diagram of a workstation computer suitable for use in various embodiments.

### DETAILED DESCRIPTION OF THE INVENTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

The term “computing device” is used herein to refer to any electronic device equipped with at least a processor and a data or network interface suitable for interfacing with a remotely-controlled catheter positioning device. The processor of a computing device may be configured with processor-executable instructions perform the operations of the embodiment methods described below.

A number of invasive medical diagnostic and treatment procedures may require more than one catheter to be concurrently utilized within an organ of a patient, such as the heart, kidneys, stomach, etc. For example, in an ablation procedure, an ablation catheter may be inserted into an organ within the patient to perform tissue ablation treatments, and an ultrasound imaging catheter may also be inserted into the same organ or an adjacent portion of the patient in order to provide imagery of the ablation catheter that clinicians may view as they manipulate the handle of the catheter in order to position the tip of the ablation catheter onto the portion of the organ to be treated. An ultrasound catheter is often used for imaging within patients because of the ability of ultrasound to image tissues through blood that will inhibit visual imaging techniques.

However, the manipulation of multiple catheters within an organ may be difficult for clinicians to manage, as the manipulations of each catheter must be precise to ensure the success of the procedure and the health of the patient. In particular, keeping the tip of a treatment catheter within the field of view of an ultrasound imaging catheter may require complex coordination of the two catheter handles. This difficulty coordinating two catheters simultaneously may add to the complexity of the procedure and the difficulty for the clinician. For example, when an ablation catheter is repositioned within a chamber of the patient's heart in order place the tip of the ablation catheter on the tissue to be ablated, the ablation catheter may move outside of the field of view of the ultrasound catheter, requiring the clinician to move the ultrasound catheter as well. Thus, a clinician may be forced to move the ablation catheter an increment, adjust the orientation of the ultrasound catheter to reacquire the ablation catheter within the ultrasound image, and repeat this process until the ablation catheter has been repositioned to a new location for treatment.

Catheter Robotics Inc. has developed a remotely-controlled catheter positioning system that is configured to enable clinicians to position catheters within a patient from a location removed from exposure to ionizing radiation. An example of such a remotely-controlled catheter positioning system is disclosed in U.S. patent application Ser. No. 13/432,161 filed on Mar. 28, 2012, entitled “Remotely Controlled Catheter Insertion System with Automatic Control System,” which is incorporated herein by reference in its entirety for disclosing details of that system. Such a remotely-controlled catheter positioning system enables clinicians to remotely maneuver, articulate, and orient catheters (generally referred to herein as “positioning” or “repositioning” catheters).

The various embodiments provide systems, devices, methods, and non-transitory processor-readable storage media for enabling a remotely-controlled catheter positioning system to automatically reposition a first catheter within a patient based on the position of a second catheter. In particular, the various embodiments may enable a remotely-controlled catheter positioning system to reposition an ultrasound imaging catheter in order to keep its imaging field of view focused on the second catheter, such as an ablation catheter. In an embodiment, a computing device coupled to or included within the control system of a remotely-controlled catheter positioning system may be configured with processor-executable instructions to analyze ultrasound imagery obtained from an ultrasound catheter in order to determine when and how the remotely-controlled catheter positioning system should move in order to reposition the ultrasound catheter so as to keep the second catheter within the ultrasound imagery. For example, when the computing device determines that an ablation catheter co-located in a patient with the ultrasound catheter is moving out of the imaging field of view, the computing device or control system may transmit control signals to the ultrasound catheter's remotely-controlled catheter positioning system to move control actuators in order to bend, translate, and/or rotate the ultrasound catheter until the ablation catheter's tip is once again centered within the field of view (i.e., at or near the center of the ultrasound image generated by the ultrasound catheter).

In an embodiment, the computing device may be configured to prompt a clinician to confirm automated movements of the ultrasound catheter before the commands are sent to the remotely-controlled catheter positioning system. For example, the computing device may render warnings, input requests, or other messages to ensure that proper professional oversight is maintained over automated catheter movements caused by the remotely-controlled catheter positioning system. Such prompts for clinician authorization before automatic movements are initiated may be rendered on a per-action basis, on a per procedure basis, or periodically.

In an embodiment, the computing device may use reports of movements by a second remotely-controlled catheter positioning system that is controlling the second catheter (e.g., the ablation catheter) in order to anticipate repositioning movements that should be implemented by the remotely-controlled catheter positioning system positioning the imaging catheter. In this embodiment, the computing device may obtain reports of control signals sent to the second remotely-controlled catheter positioning system for positioning the second catheter (e.g., the ablation catheter), anticipate a compensatory movement that may be required for the imaging catheter, and generate control signals for the remotely-controlled catheter positioning system controlling the ultrasound catheter based so that the imaging field of view moves in parallel with movements of the second catheter. The control signals sent to the second remotely-controlled catheter positioning system may be from a clinician or automated (e.g., preprogrammed) movement commands issued by a computing device controlling the second remotely-controlled catheter positioning system. For example, the computing device may receive indications of movement commands executing by the second remotely-controlled catheter positioning system to reposition an ablation catheter, determined that the resulting movement of the ablation catheter will take it out of the ultrasound catheter's imaging field of view, and issue commands to the remotely-controlled catheter positioning system controlling the ultrasound catheter to move in a compensatory manner. As another example, when the second remotely-controlled catheter positioning system is executing a preprogrammed sequence of movements to reposition an ablation catheter according to a prescribed procedure (e.g., to achieve a pre-defined ablation pattern), the computing device may issue commands to the remotely-controlled catheter positioning system controlling the ultrasound catheter to maintain the ablation catheter within the field of view without any inputs by the clinician supervising the preprogrammed ablation treatment.

The computing device may further be configured to refine movements of the remotely-controlled catheter positioning system positioning the ultrasound catheter that are performed in response to movement commands issued to the second remotely-controlled catheter positioning system by evaluating subsequently obtained ultrasound imagery and adjusting the algorithm used to determine compensatory movements. In this manner, the computing device may learn how the ultrasound catheter is responding to movement commands (which may depend upon the particular orientation of the catheter at the time) sees, and adjust the compensatory movement commands in order to better maintain the ablation catheter within the imaging field of view.

In various embodiments, ultrasound imagery may be evaluated by the computing device to identify contextual or characteristic information about an organ in which a procedure is being perform, and use such information to change, correct, or modify control algorithms used to generate movement commands to the remotely-controlled catheter positioning system controlling the ultrasound imaging catheter. For example, when the computing device determines that a certain translation of the ultrasound catheter would cause its distal end (which includes the transducer) to collide with tissue (e.g., a heart wall or valve) or otherwise degrade resulting imagery, the computing device may adjust the translation instructions to avoid such a situation.

The remotely-controlled catheter positioning systems may be configured to position various types of catheters and therefore the various embodiments are not limited to ablation catheters and ultrasound catheters that are referred to herein as example embodiments. Further, although the various embodiments may be described as utilizing ultrasound data and/or ultrasound imagery, it should be appreciated that other forms of data and imagery from imaging catheters may be utilized by the various embodiments.

The descriptions of various embodiments refer generally to a “computing device” performing operations of processing imagery and determining catheter repositioning movements that should be executed by a remotely-controlled catheter positioning system. Such a computing device may be a separate computer, part of the control system for a remotely-controlled catheter positioning system, and/or part of an imaging system, such as integrated within an ultrasound catheter processing system.

In embodiments in which the operations attributed to the computing device are performed in a separate computer, such as a portable or workstation computer, (i.e., a computing device that is not part of either the control system for a remotely-controlled catheter positioning system or the imaging system), that separate computer may be coupled via data and command communication links (i.e., data cables and/or wireless data links) to each of the control system for a remotely-controlled catheter positioning system and the imaging system.

In embodiments in which the operations attributed to the computing device are performed within the control system for a remotely-controlled catheter positioning system, the computing device may be implemented as a software module executing within the control system computer (i.e., the control system computer and the computing device may be the same processor executing different software), or as a co-processor or second computer within the same control system (i.e., the control system computer and the computing device may be separate processors within the same system).

In embodiments in which the operations attributed to the computing device are performed within the imaging system, such as an ultrasound catheter processing system, the computing device may be implemented as a software module executing within the imaging system computer (i.e., the imaging system computer and the computing device may be the same processor executing different software), or as a co-processor or second computer within the imaging system (i.e., the imaging system computer and the computing device may be separate processors within the same system).

In further embodiments, some of the operations of the embodiment methods may be performed in the imaging system computer (e.g., processing ultrasound images) and other operations may be performed in a separate computer or within the control system of a remotely-controlled catheter positioning system.

For ease of reference, these various alternative configurations for the computer performing the embodiment methods are referred to generally herein as being performed in a “computing device.” Similarly, a generic computer is shown in the system drawings referenced in the embodiment descriptions that follow. However, the use of this general term in the descriptions of the embodiments is not intended to limit the scope of the claims to a particular embodiment or configuration unless a specific configuration is recited in the claims themselves.

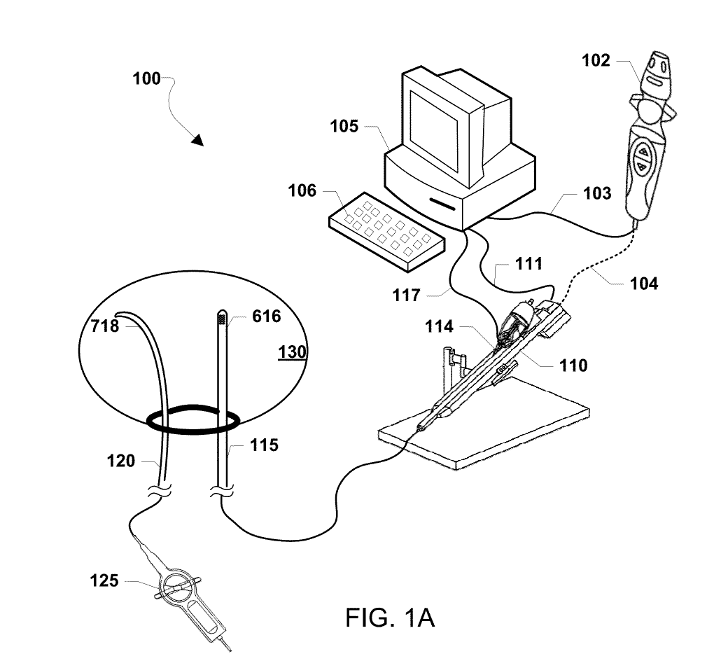

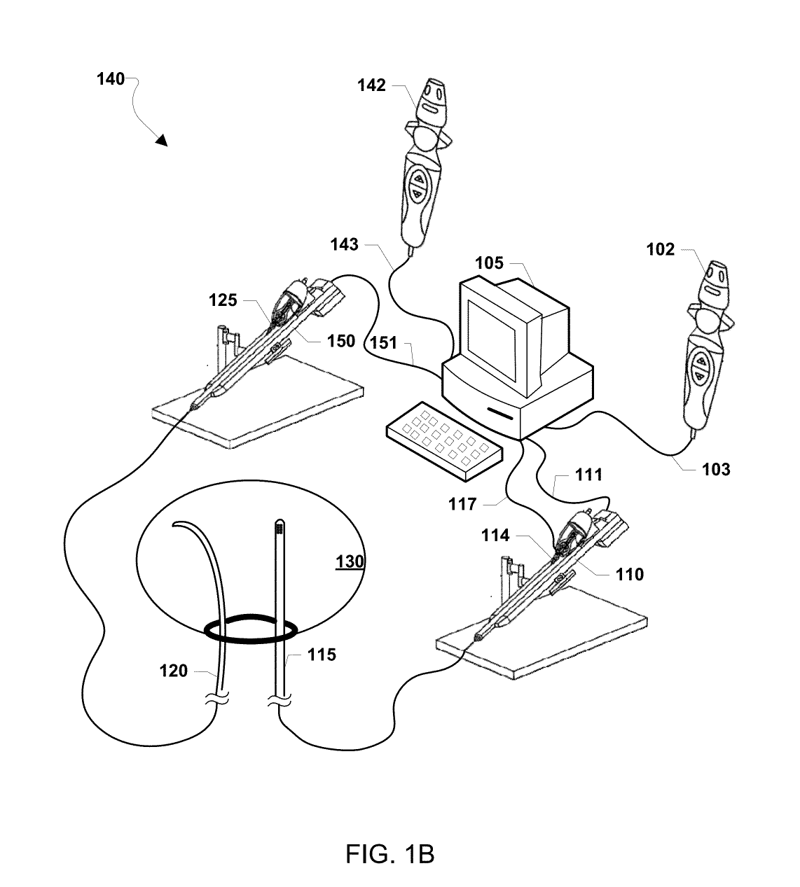

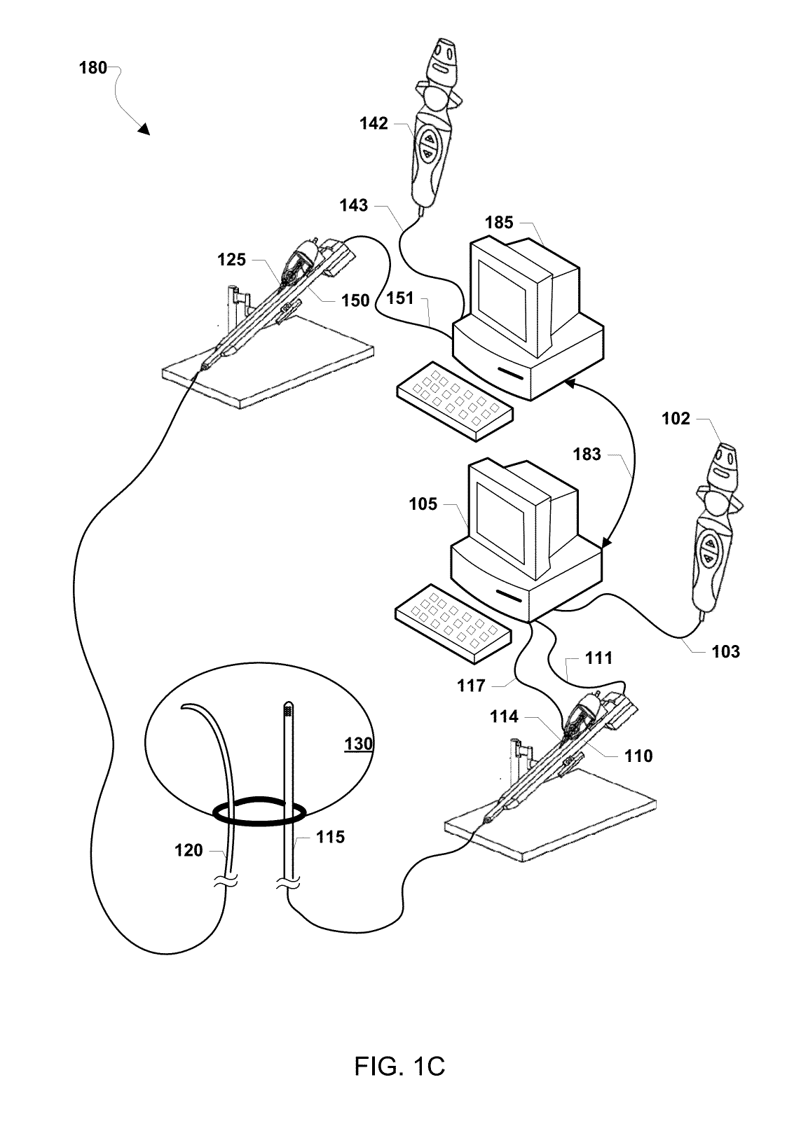

FIG. 1A illustrates an embodiment remotely-controlled catheter positioning system 100 that includes a computing device 105 in communication with at least a remote controller 102 and a remotely-controlled catheter positioning system 110. The computing device 105 may be a programmable device configured to generate instructions and transmit signals for controlling the insertion and positioning of catheters by the remotely-controlled catheter positioning system 110. The remote controller 102 may be connected to the computing device 105 via a wired connection 103, and the remotely-controlled catheter positioning system 110 may be connected to the computing device 105 via a wired connection 111. In various embodiments, any of the devices 102, 105, and 110 may be connected via wireless links (not shown), such as WiFi, WiFi Direct, Bluetooth, NFC, or any other wireless signaling protocol capable of transmitting signals between the various devices 102, 105, 110 within predefined latency tolerance thresholds. For example, the remote controller 102 may communicate with the computing device 105 via Bluetooth signaling; however, the remotely-controlled catheter positioning system 110 may be configured to receiving signaling from the computing device 105 via the wired connection 111. In an embodiment, the remote controller 102 may be configured to communicate directly with the remotely-controlled catheter positioning system 110 via a wired or wireless connection 104.

An ultrasound catheter 114 (or other imaging catheter) may be installed on or attached to the remotely-controlled catheter positioning system 110. Based on the control signals received from the computing device 105 via the connection 111, the remotely-controlled catheter positioning system 110 may manipulate the ultrasound catheter 114 within a patient, such as by advancing, retracting, and/or rotating the ultrasound catheter 114, as well as actuating or bending a tip of the tubular member 115 by actuating a control manipulator on the catheter handle. Thus, based on actions of the remotely-controlled catheter positioning system 110, a tubular member 115 of the ultrasound catheter 114 may be manipulated within the patient (e.g., a human, horse, various mammals, etc.) so that its distal end 215 is positioned within an organ 130 of the patient. For example, the organ 130 may be a blood vessel or a chamber of the patient's heart. A tubular member 120 of a second catheter 125 (e.g., an ablation catheter) may be manipulated within the subject such that its distal end 220 is also positioned within the organ 130. In an embodiment, the second catheter 125 may be manipulated by hand (e.g., manually inserted and positioned by a physician manipulating the catheter handle) or alternatively by a second remotely-controlled positioning device, as described below with reference to FIGS. 1B and 1C.

In an embodiment, the computing device 105 may be configured to exchange data (e.g., the ultrasound image data obtained by the ultrasound catheter 114) with the ultrasound catheter 114 via a connection 117. For example, the ultrasound catheter 114 may collect and relay (or transmit) raw ultrasound echo data to the computing device 105 to be processed into ultrasound imagery that may be viewed by a clinician and/or evaluated by the computing device 105. In another embodiment, the computing device 105 may receive ultrasound data from another device, such as a dedicated ultrasound data processing system (not shown separately) connected to the ultrasound catheter 114.

In an embodiment, the computing device 105 may store pre-programmed commands or instructions that may be transmitted to the remotely-controlled catheter positioning system 110, such as transmitting control signals for a pre-programmed movement (e.g., rotational and translational movements, a corkscrew maneuver, etc.) in response to a user input, such as a user selecting a file name and pressing an execute key on the remote controller 102 or the system keyboard 106. For example, an embodiment remote controller 102 may include a single button that may be pushed to activate a pre-programmed sequence of operations (e.g., a corkscrew maneuver) that includes rotational and translational movement commands programmed and stored in the computing device 105. Pre-programmed movement commands may also be executed in response to feedback (e.g., sensor data, ultrasound data, etc.) received from the remotely-controlled catheter positioning system 110 and/or the catheter controlled by the remotely-controlled catheter positioning system 110. For example, ultrasound data from the ultrasound catheter 114 controlled by the remotely-controlled catheter positioning system 110 may be received at the computing device 105 and processed to trigger automated or pre-programmed movements. As another example, a pressure sensor on the tip on the catheter may send signals to the computing device 105 when the catheter tip presses against tissue, such as signals indicating an amount of pressure being applied to tissue, which may trigger an automated movement of the remotely-controlled catheter positioning system 110 to relieve that pressure.

FIG. 1B illustrates another embodiment catheter positioning system 140 that includes a computing device 105 configured to two remotely-controlled catheter positioning systems 110, 150. In this embodiment, the computing device 105 may be in communication with more than one remote controller 102, 142 via connections 103, 143 and more than one remotely-controlled catheter positioning system 110, 150 via connections 111, 151. For example, the computing device 105 may be configured to receive remote control command signals from the first remote controller 102 (e.g., positioning actions from a physician operator, etc.) via the connection 103 and, in response, transmit control signals via the connection 111 to the first remotely-controlled catheter positioning system 110 to move and/or manipulate a tubular member 115 of an ultrasound catheter 114 within an organ 130. Further, the computing device 105 may be configured to receive remote control data from the second remote controller 142 via the connection 143 and, in response, transmit control signals via the connection 151 to the second remotely-controlled catheter positioning system 150 to move a tubular member 120 of a second catheter 125 (e.g., an ablation catheter, etc.) that is also within the organ 130. In an embodiment, the remote controllers 142, 102 may be operated by a single clinician or by separate clinicians, such as a first clinician performing the ablation procedure while a second clinician manipulates the ultrasound imaging catheter to provide suitable images of the treatment site. For example, a physician may first use the second remote controller 142 to enter inputs to cause the second catheter 125 to be moved within the organ 130 and then use the first remote controller 102 to provide inputs to cause the ultrasound catheter 114 to be positioned differently in the organ 130.

The computing device 105 may be configured to receive data generated by or related to both catheters 114, 125, such as sensor data related to the manipulation of the catheters within the organ 130 and/or imaging data. The computing device 105 may be configured to execute separate, concurrent threads, routines, applications, software, or other instructions to properly handle the data exchanges. For example, the computing device 105 may utilize concurrently executing threads for monitoring buffers associated with feedback data (or imaging data) from the individual catheters 114, 125 and/or equipment related to the catheters 114, 125, such as a separate device for buffering, formatting, or otherwise processing data from sensors within the catheters 114, 125.

As described above, data received by the computing device 105 from the ultrasound catheter 114 may be used to generate repositioning instructions and control signals for a remotely-controlled catheter positioning system to cause repositioning of the ultrasound catheter 114 within the patient. In particular, ultrasound data obtained by the ultrasound catheter 114 may be received and processed by the computing device 105 to generate repositioning instructions and control signals that cause the first remotely-controlled catheter positioning system 110 to manipulate the ultrasound catheter 114 so that its transducer may be better positioned to obtain the desired ultrasound data/imagery (e.g., images of the second catheter).

As mentioned above, the automated repositioning of the catheter initiated by the computing device 105 may also be based, at least in part, upon input signals received from the second remote controller 142. In particular, the computing device 105 may receive inputs from the second remote controller 142 for the second remotely-controlled catheter positioning system 150, evaluate the repositioning of the second catheter 125 that may result from the inputs, and generate repositioning instructions and control signals for the first remotely-controlled catheter positioning system 110 based on the received inputs for the second remotely-controlled catheter positioning system 150. In this way, a single clinician may be assisted by the computing device 105 to facilitate moving both catheters 114, 125 in tandem based on the clinician's input commands entered on only one of the remotely-controlled catheter positioning systems 110, 150. In other words, an embodiment computing device 105 may use repositioning commands being executed by a remotely-controlled catheter positioning system positioning an ablation catheter 125 to generate control commands for the remotely-controlled catheter positioning system positioning the ultrasound catheter 114 so that the ultrasound catheter 114 is moved automatically in tandem an appropriate or compensatory amount to ensure that imaging of the ablation catheter 125 is maintained.

Regardless of any dependent repositioning instructions, the computing device 105 may be configured to cause the ultrasound catheter 114 to be repositioned in response to clinician inputs received from the first remote controller 102. In other words, the computing device 105 may control the remotely-controlled catheter positioning systems 110, 150 so that the ultrasound catheter 114 may be moved automatically based on the repositioning of the second catheter 125 and manually based on manual inputs. This embodiment allows the ultrasound catheter 114 to be moved when the second catheter 125 is moved, but also allows the clinician to make fine adjustments (or corrections) via the first remote controller 102.

FIG. 1C illustrates another embodiment catheter positioning system 180 that is similar to the system 140 described above, except that the system 180 includes a second computing device 185 that is associated with the second remotely-controlled catheter positioning system 150 and second remote controller 142. In this embodiment, the second computing device 185 and the first computing device 105 may be configured to exchange data with each other via a wired or wireless connection 183 (e.g., a direct or Peer-to-Peer link, a radio connection, a link via a local area network, etc.) that can be used for automatically repositioning one or both of the catheters. In other words, instead of processing all inputs and data exchanges associated with the first and second remotely-controlled catheter positioning systems 110, 150, remote controllers 102, 142, and catheters 114, 125, the first computing device 105 may be dedicated to controlling the ultrasound catheter 114 via control signals to the first remotely-controlled catheter positioning system 110. The first computing device 105 may receive data from the second computing device 185 via the data connection 183 between them, which may include exchanging positioning commands issued to their respective remotely-controlled catheter positioning systems and exchanging data received from their respective catheters. Each of the first and second computing devices 105, 185 may be configured to utilize data received from the other computing device to automatically generate repositioning commands for their respective remotely-controlled catheter positioning systems.

As an example, in response to receiving a control input via the second remote controller 142, the second computing device 185 may transmit control signals to the second remotely-controlled catheter positioning system 150 to move the second catheter 125 within the organ 130. At the same time, the second computing device 185 may also report such control signals to the first computing device 105 via the data connection 183 between them. In response to receiving such control signals, the first computing device 105 may generate repositioning instructions and control signals for the first remotely-controlled catheter positioning system 110 to move the ultrasound catheter 114 within the patient in a compensatory manner.

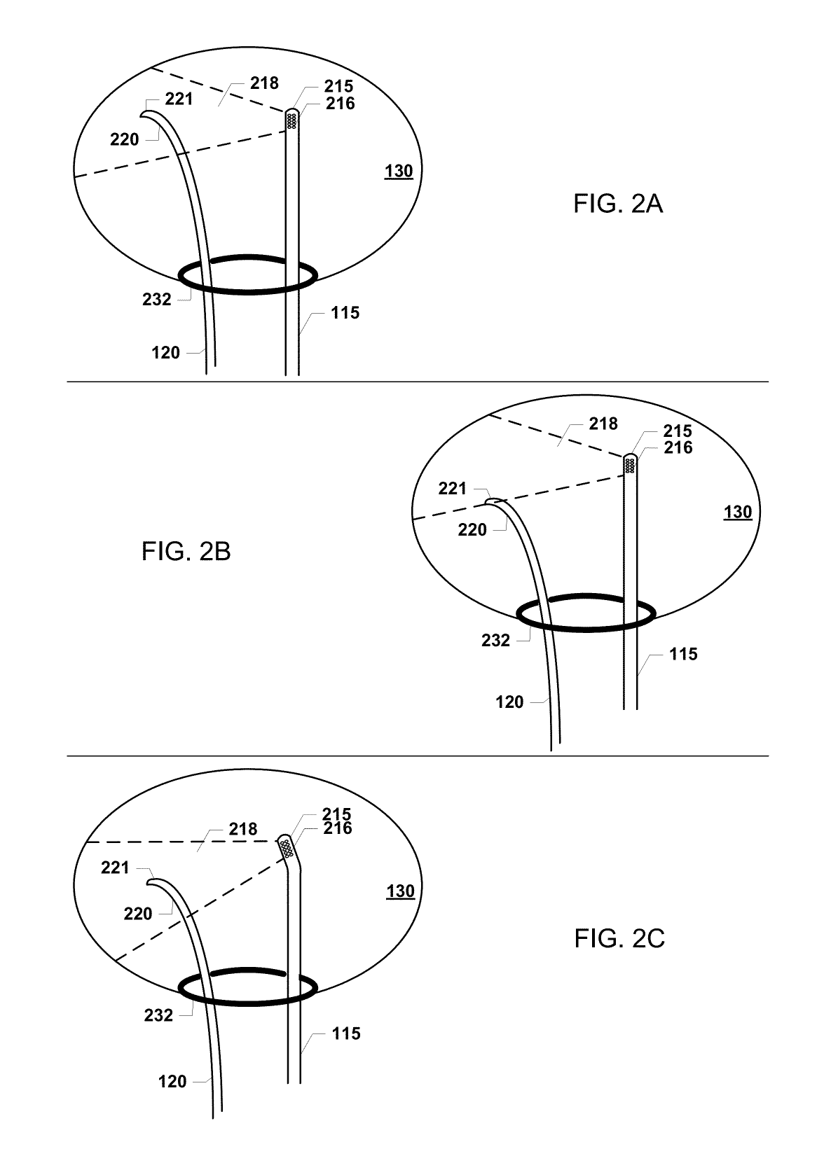

FIGS. 2A-2C illustrate distal ends 215, 220 of two catheters in various states of positioning within an organ 130 in order to illustrate automated catheter movements enabled by the various embodiments. The first distal end 215 may be located at the end of a first tubular member 115 of an ultrasound catheter configured to be repositioned via a remotely-controlled catheter positioning device, such as shown above with reference to FIGS. 1A-1C. The first distal end 215 may include an ultrasound transducer 216 configured to transmit ultrasound and receive ultrasound echoes, and translate the received echoes into electrical signals that are transmitted through the catheter to an ultrasound imaging system which can generate imagery of the field of view imaging volume 218. The imaging volume 218 represents the three-dimensional volume within the organ 130 that can be imaged by the ultrasound catheter and includes the imaging field of view to the depth at which ultrasound echoes becomes too attenuated to be received. An example of an ultrasound catheter with a transducer and related components is described in greater detail below.

The second distal end 220 may be located at the end of a second tubular member 120 of a second catheter, such as an ablation catheter. The second catheter may be repositioned via a remotely-controlled catheter positioning device, such as shown in the FIGS. 1A-1C above. Both distal ends 215, 220 may have been positioned through an entry 232 to the organ 130, such as being guided through a vein or artery into a chamber of the patient's heart (e.g., a ventricle, atrium, etc.).

In the example shown in FIG. 2A, the first distal end 215 has been be positioned so that the imaging volume 218 includes the distal end 220 of the second catheter. In other words, ultrasound imagery produced from ultrasound data corresponding to the imaging volume 218 will include at least the distal end 220 of the second catheter. Further, the tip 221 of the distal end 220 of the second catheter may be located in the center (or middle) of the imaging volume 218, and therefore may be considered well represented within the result ultrasound imagery. It may be beneficial to manipulate the ultrasound imaging catheter so that the tip 221 of the ablation catheter remains within the center of the imaging volume 218 as equipment or other functionality within the tip 221 (e.g., electrode, laser, etc.) may need to be seen for performing various procedures within the organ 130. For example, a physician may need to have a clear view of an ablation electrode in the tip 221 in order to ablate the correct surface tissue within a heart.

For the purposes of this disclosure, the center of an image may be considered an optimal position (or a “desired location”) for an object to be viewed within imagery. However, other locations within imagery may be preferred for various procedures, clinicians, and/or equipment. To accommodate this, particular desired locations within ultrasound images (e.g., target regions or coordinates within imagery) may be stored within a computing device as procedure, equipment or clinician preferences, such as within stored profiles corresponding to clinicians, procedures, patients, etc.

In the example illustrated in FIG. 2B, a clinician has repositioned the second catheter so that the distal end 220 and the tip 221 of the second catheter has moved outside of the center of the imaging volume 218, causing limited visibility of the second catheter within the resulting ultrasound imagery. With the tip 221 in a suboptimal or non-preferred location in the ultrasound imagery, the clinician may not be able to properly see the parts of the organ 130 that are relevant to the operations of the second catheter, and thus may have limited effectiveness and precision.

In the example illustrated in FIG. 2C, the distal end 215 of the first catheter has been repositioned (e.g., bent) so that the tip 221 of the distal end 220 of the second catheter is included within the center of the imaging volume 218. As described below, the repositioning of the first catheter may have been performed by a remotely-controlled catheter positioning system in response to receiving control signals from a computing device that has processed ultrasound data as received by the transducer 216. Alternatively, the distal end 215 of the first catheter may have been translated (e.g., pushed/pulled forward, backward, sideways), rotated at one or more axis (e.g., corkscrewed, etc.), or otherwise contorted based on the structure and manipulation capabilities of the first catheter.

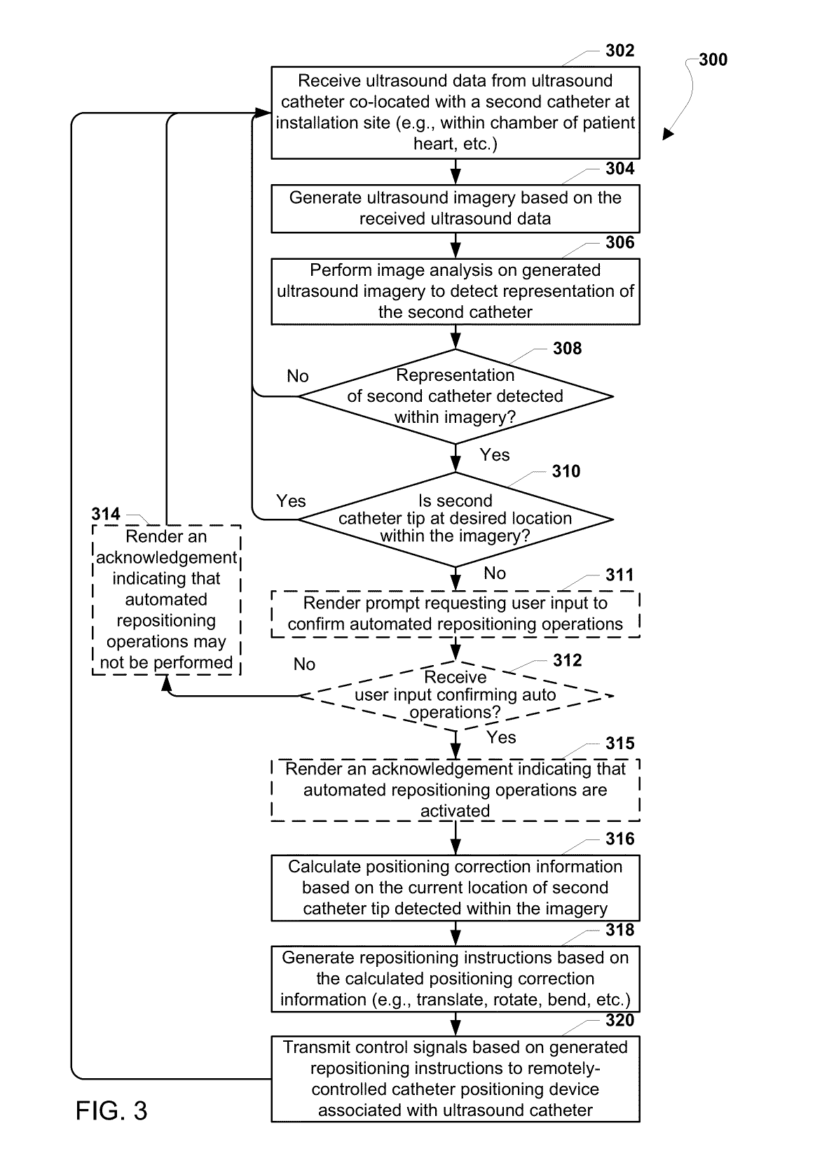

FIG. 3 illustrates an embodiment method 300 for a computing device to generate repositioning instructions for a remotely-controlled catheter positioning system to automatically reposition an ultrasound catheter based on ultrasound data received from the ultrasound catheter. As described above, the computing device may be configured to perform software, applications, routines, and various operations to determine how an ultrasound catheter should be repositioned (e.g., translated, rotated, bent, etc.) via a remotely-controlled catheter positioning system in order to maintain a target catheter within ultrasound imagery. In particular, the computing device may evaluate ultrasound imagery generated by the ultrasound catheter to detect images of a second catheter (e.g., ablation catheter, etc.) that is being used in a procedure, such as a surgery or other medical operation. As the second catheter may be the primary implement for the procedure, the ultrasound catheter may be needed to image the second catheter to enable the clinician to successfully conduct the procedure. When a target catheter is not detected within the ultrasound imagery in a satisfactory manner (e.g., out of frame, poor angle of view, etc.), the computing device may generate control commands for the remotely-controlled catheter positioning system to move the tip of the ultrasound catheter into a position that better images the target catheter. For example, when the computing device determines that the ablation catheter tip is not present in the ultrasound imagery, the computing device may transmit control signals to the remotely-controlled catheter positioning system to cause the ultrasound catheter to be re-oriented so that the tip of the ablation catheter appears in or near the center of the ultrasound imagery.

In block 302, the computing device may receive ultrasound data from the ultrasound catheter co-located with a second catheter within an organ (e.g., within chamber of patient heart, etc.). The computing device may receive the ultrasound data from the ultrasound catheter directly when the computing device is directly coupled to the ultrasound catheter or from an ultrasound imaging system, which receives the ultrasound data from the ultrasound catheter and renders ultrasound images. For example, the computing device may receive the ultrasound data after it has been stored, formatted, and otherwise processed by a dedicated ultrasound imaging system directly coupled to the ultrasound catheter. In an embodiment, the computing device may receive the ultrasound data or ultrasound images from a storage device or other data structure configured to buffer, store, or maintain ultrasound data.

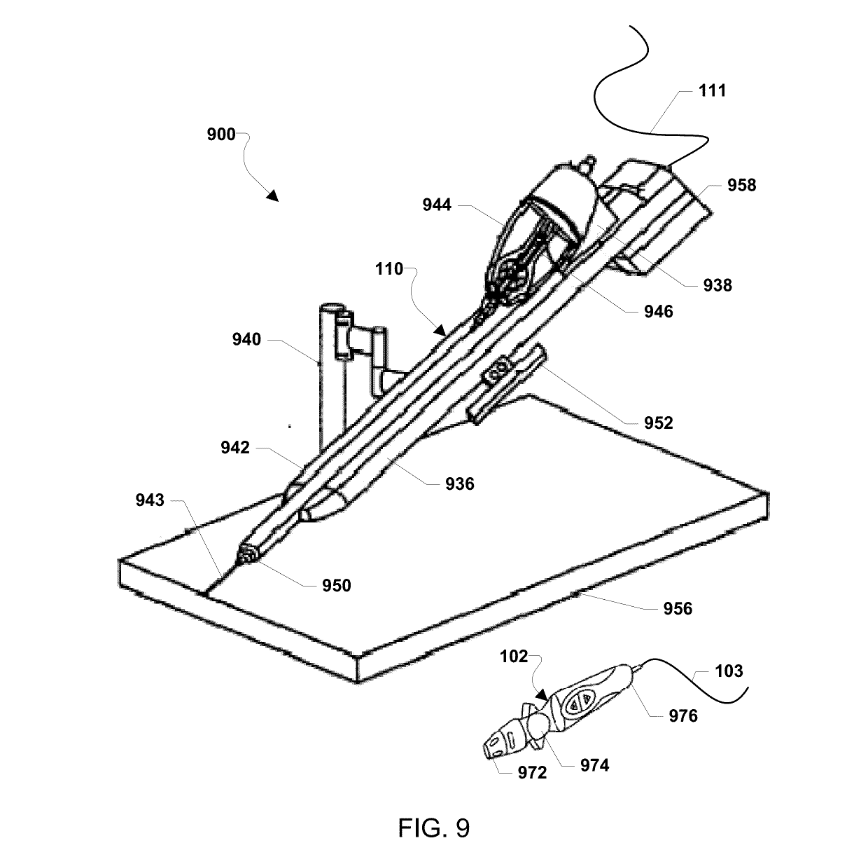

The ultrasound catheter may be any catheter type, structure, or design capable of relaying ultrasound data for imaging the organ and capable of being positioned within the organ by a remotely-controlled catheter positioning device, such as described below with reference to FIG. 9. Further, the second catheter may be any catheter type, structure, or design capable of being used in various procedures or operations, such as ablation procedures. In an embodiment, the second catheter may also be capable of being positioned within the organ by a remotely-controlled catheter positioning device.

In optional block 304, the computing device may generate ultrasound imagery based on the received ultrasound data in embodiments in which the ultrasound data is processed directly by the computing device (i.e., not by an ultrasound imaging system). The computing device may perform various decoding, conversion, and processing operations, routines, or software to generate ultrasound imagery that may provide graphical representations of the organ based on the received ultrasound data. For example, the computing device may perform a software routine that converts the received ultrasound data into a static image or a series of images (e.g., a video file). In an embodiment, the computing device may receive ultrasound imagery from another device, such as a dedicated ultrasound processing device.

The computing device may perform image analysis operations on the ultrasound imagery to detect the presence of the second catheter within the image volume in block 306. Such image analysis operations may include evaluating the visual characteristics of the various segments of the generated imagery, such as identifying areas with high or low contrast or reflectivity, movement over a time period, changing values (e.g., blinking, etc.), predefined shapes, deformations over a time period, etc. In an embodiment the computing device may be configured to filter out irrelevant information within the ultrasound imagery, such as motion blur or artifacts caused by corrupt data or other errata. Further, the computing device may be configured to analyze the ultrasound imagery based on the organ structure, such as by utilizing predefined thresholds for determining errata or movement that are informed by typical conditions in the organ (e.g., typical heartbeat frequency ranges, tissue densities in a certain area of vascular system, etc.).

In an embodiment, the second catheter may include one or more ultrasound reflectors (e.g., microspheres or bubbles embedded within the catheter) configured to produce a loud echo (which may appear as a bright spot in the ultrasound imagery) affixed to or near the tip of the catheter to function as a recognizable features or fiducial marks to facilitate locating the second catheter within ultrasound images. In this embodiment, the computing device may be configured to evaluate the ultrasound data to identify and track such ultrasound reflectors, use the resulting loud echoes is fiducial marks for tracking the catheter tip. For example, the computing device may analyze the ultrasound imagery to determine whether an ultrasound reflector known to be located at the tip of the second catheter (e.g., an ultrasound reflector) appears within the ultrasound imagery. In an embodiment, such an element at the tip may be made of a particular material or surface known to be highly reflective regarding ultrasound waves or otherwise cause highly recognizable representations within ultrasound imagery.

In determination block 308, the computing device may determine whether the second catheter is detected within the imagery based on the image analysis. For example, the computing device may determine whether any segment within the ultrasound imagery matches the approximate shape, consistency, materials, transparency, and/or size of a man-made object resembling a catheter. In an embodiment, the computing device may perform pattern-matching operations, such as by comparing segments within the analyzed ultrasound imagery to predefined images of catheters (e.g., pictures of a catheter tip or lumen, etc.). The computing device may also compare the ultrasound imagery to information known about the second catheter, such as shapes, ultrasound reflectors, and echo characteristics. For example, the computing device may use stored data regarding the expected density and tip shape of the second catheter when processing the ultrasound imagery to recognize and locate the tip of the catheter within an ultrasound image. In various embodiments, the computing device may utilize tolerance thresholds in its determinations and may only determine that the second catheter is imaged within the ultrasound imagery when the analysis falls within a certain confidence or certainty threshold.

If the computing device determines that the second catheter is not imaged within the ultrasound imagery field of view (i.e., determination block 308=“No”), the computing device may repeat the operations of receiving ultrasound images in block 302 to continuing looking for the presence of the second catheter in the images. In this situation, the computing device may execute a preprogrammed to search routine by which it causes the ultrasound catheter tip to move in a preprogrammed sequence to scan a large volume in order to find the target catheter. Alternatively, the computing device may process images while a clinician manipulates a controller and sending movement commands to the remotely-controlled catheter positioning system. A clinician not seeing the second catheter in the ultrasound imagery may command the remotely-controlled catheter positioning system so as to reposition the ultrasound catheter searching for the target catheter. For example, a doctor conducting a procedure with an ablation second catheter may interact with a remote controller to reposition the ultrasound catheter until the ablation catheter can be seen to some degree within the resulting ultrasound imagery. Thus, operations of blocks 302 through 308 may continue in a loop while the clinician manipulates the ultrasound catheter via its corresponding remotely-controlled catheter positioning system until the second catheter is within view of the ultrasound imagery (i.e., determination block 308=“Yes”).

When the computing device determines that the second catheter is within the ultrasound image volume (i.e., determination block 308=“Yes”), the computing device may determine whether the second catheter tip appears at a desired location within the imagery (e.g., at or near the center of the ultrasound imagery) in determination block 310. For example, the computing device may evaluate the ultrasound imagery to detect shapes associated with tips (e.g., hard, pointed objects, etc.) and determine the distance that such shapes appear from the centerline of the ultrasound image volume. As described above, the desired location of the target catheter within the ultrasound image may be predefined, such as within a stored preference or profile corresponding to the clinician, procedure, patient, and/or equipment. Tracking of enhanced ultrasound echo structures, such as microspheres within the catheter near the tip, may facilitate the accurate determination of the tip of the catheter with respect to the centerline of the ultrasound image volume.

If the computing device determines that the second catheter tip appears at or near the desired location within the ultrasound imagery (i.e., determination block 310=“Yes”), no adjustment in the positioning of the ultrasound catheter tip is necessary, so the computing device may continue with the operations of receiving ultrasound data in block 302 and processing the ultrasound to locate the catheter within.

If the computing device determines that the second catheter tip is at the desired location within the imagery (i.e., determination block 310=“Yes”), in optional block 311 the computing device may render a user prompt requesting the clinician to make an input to confirm that automated repositioning operations may begin. In other words, to ensure complete control and awareness of clinicians over automated actions for positioning a catheter within a patient, the computing device may request that the clinician approve when such automated actions are authorized to be performed. In this way, the computing device may be constrained from manipulating the ultrasound catheter except when explicitly authorized to do so. The prompt may be rendered in various ways, such as a dialog box is on a display connected to the computing device, as a predefined sound or audible cue broadcast via a speaker connected to the computing device, and/or a tactile signal, such as a rumble on the remote controller for the remotely-controlled catheter positioning device connected to the computing device.

In another embodiment, the operations in optional block 311 may be performed by the computing device after the operations in block 316 or block 318 so that more information may be presented to the clinician. For example, the computing device may render a prompt that indicates a proposed amount of repositioning of an ultrasound catheter based on the calculated positioning corrections (e.g., “OK to extract ultrasound catheter by a millimeter?”) and/or the generated repositioning instructions (e.g., “OK to bend distal end to the left by a 1 degree angle?”). In an embodiment, the computing device may render a prompt requesting permission to lock a setting for performing automated repositioning operations (e.g., “OK to activate setting for automatically repositioning ultrasound catheter?”).

In optional determination block 312, the computing device may determine whether a user input confirming the automated repositioning operations has been received. The computing device may monitor input buffers, interactions with user interfaces (e.g., a GUI button press, etc.), input gestures, and/or input from various connected peripherals, devices, or functionalities. For example, the computing device may receive a mouse click on a portion of a rendered dialog box associated with an ‘accept’ or ‘reject’ graphical user interface button. As another example, the computing device may receive audio data from a microphone and perform speech analysis to determine whether the physician has audibly confirmed automated operations (e.g., “Yes” or “Move the ultrasound catheter,” “No,” etc.). If the computing device determines a user input confirming the automated repositioning operations be performed has not been received (i.e., optional determination block 312=“No”), in optional block 314 the computing device may render an acknowledgement (e.g., visual, sound, etc.) indicating that automated repositioning operations may not be performed (e.g., a dialog box rendered on the computing device display indicating “Automated operations are deactivated,” etc.), the computing device may continue receiving ultrasound data in block 302.

If the computing device determines that a user input confirmed conducting automated repositioning operations (i.e., optional determination block 312=“Yes”), in optional block 315 the computing device may render an acknowledgement indicating that automated repositioning operations are activated (e.g., a dialog box rendered on the computing device display indicating “Automated repositioning active.”).

In an embodiment, the operations in optional block 311 and optional determination block 312 may be performed repeatedly, periodically, or until reset. For example, to avoid redundant or distracting prompts, the computing device may be configured to only prompt a clinician to confirm whether to allow automated repositioning operations once every predetermined time period (e.g., prompt once every few seconds, minute, etc.), or alternatively based on a predefined event (e.g., prompt once per procedure, after a predefined number of control signals are transmitted, etc.). In an embodiment, the computing device may be configured to prompt the user to confirm automated repositioning whenever the second catheter comes into view within the ultrasound imagery after being absent from the imagery.

In block 316, the computing device may calculate positioning corrections based on the current location of the second catheter tip detected within the imagery. For example, the computing device may calculate a lateral distance (e.g., a number of millimeters, etc.) to move the ultrasound catheter based on the distance and direction from the center of the current ultrasound imagery to the tip of the second catheter. The computing device may also calculate orientations and various other movements that may need to be performed in order to center-align subsequent ultrasound imagery to the tip of the second catheter.

In block 318, the computing device may generate repositioning instructions based on the calculated positioning corrections (e.g., translate, rotate, bend, etc.). In other words, the computing device may identify actions that may be performed by a remotely-controlled catheter positioning system to manipulate the ultrasound catheter based on the positioning correction information. For example, the computing device may determine that instructions employing a certain amount of pushing (or pulling) may be performed in order to correct for the second catheter's tip being offset laterally from the center of the current ultrasound imagery. As another example, the computing device may determine that an instruction for rotating a certain number of degrees may be performed by the remotely-controlled catheter positioning system to place the second catheter's tip in the middle of subsequent ultrasound imagery. The instructions may be API commands or other instructions that utilized a predefined set of operations that may be performed by the remotely-controlled catheter positioning device.

In an embodiment, the computing device may utilize predefined data describing the organ when generating the repositioning instructions in block 318. For example, the computing device may compare the current location of the ultrasound catheter to model data representing the organ (e.g., a model of the geography of the heart, etc.) to determine whether generated instructions would cause a collision with the anatomy of patient's organ. In this way, the computing device may modify (e.g., decrease or increase the values of movements) the generated instructions, or alternatively delete instructions entirely, when such actions would not safe for the patient. Additionally, the computing device may use such predefined data to avoid generating instructions that may cause the ultrasound catheter to be poorly positioned and/or unable to relay useful ultrasound data. For example, the computing device may evaluate data representing the organ to determine whether the generated instruction would move the ultrasound catheter in such a way that its data collection would be impeded, and if so, adjust the generated instructions. In an embodiment, the predefined data may be general data (e.g., anatomical data for a typical heart, etc.) or alternatively may be specific to the particular patient (e.g., data of Mr. Smith's actual organs, etc.).

In block 320, the computing device may transmit control signals based on the generated repositioning instructions to a remotely-controlled catheter positioning system associated with the ultrasound catheter. In other words, the computing device may communicate the generated instructions to the remotely-controlled catheter positioning system in order to cause the repositioning of the ultrasound catheter. The control signals may be formatted for use by remotely-controlled catheter positioning systems as described below with reference to FIG. 9. The foregoing operations may be repeated in a continuous process by the computing device continuing to receive ultrasound images in block 302.

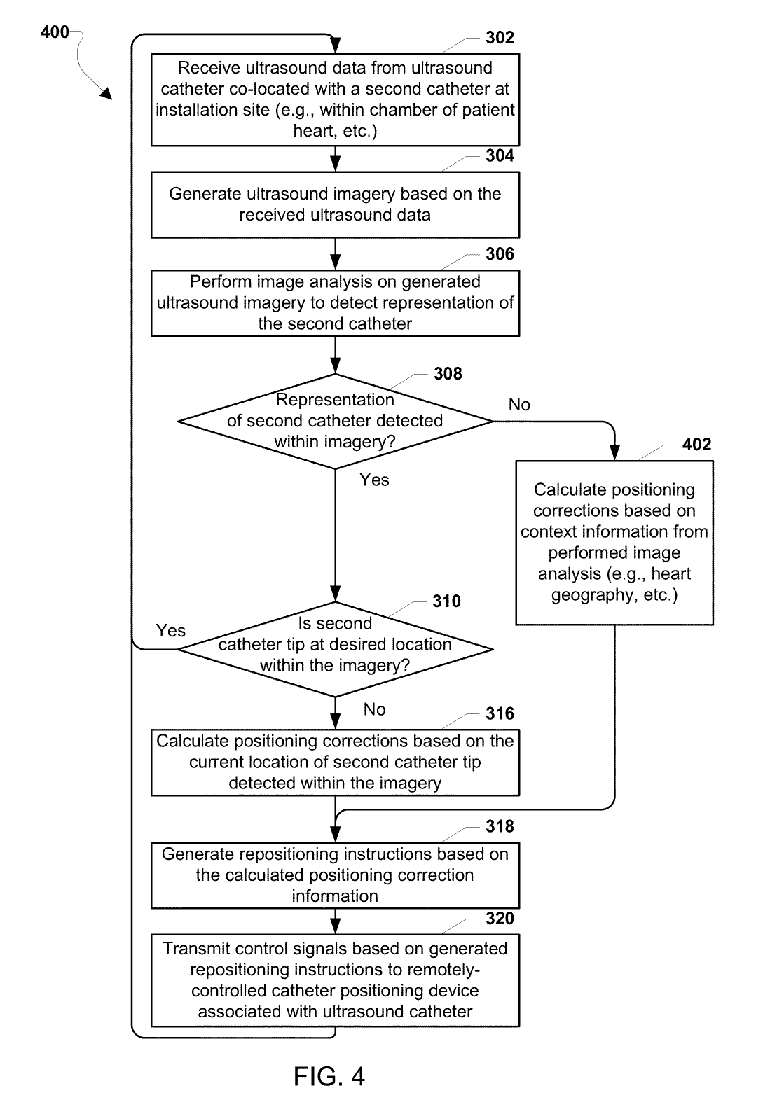

FIG. 4 illustrates an embodiment method 400 for a computing device to generate repositioning instructions for a remotely-controlled catheter positioning system to position an ultrasound catheter based on ultrasound data from the ultrasound catheter. The method 400 is similar to the method 300 described above, except that the method 400 also includes operations for generating repositioning instructions and related control signals when the second catheter is not detected within ultrasound imagery. For example, instead of merely restarting a feedback loop in response to not detecting the second catheter within ultrasound imagery, the computing device may perform operations to identify movements of the ultrasound catheter that may put the second catheter in view. After the second catheter is placed in view, the computing device may perform additional operations that may bring the tip of the second catheter closer to the center of the view. In other words, the computing device may automatically and iteratively perform operations to cause the ultrasound catheter to be placed in more improved positions for viewing the second catheter within a patient's organ, regardless of an initial lack of imagery representing the second catheter.

In block 302, the computing device may receive ultrasound data from an ultrasound catheter co-located with a second catheter within an organ (e.g., within chamber of patient heart, etc.). In block 304, the computing device may generate ultrasound imagery based on the received ultrasound data. In block 306, the computing device may perform image analysis operations on the generated ultrasound imagery to detect a representation of the second catheter. In determination block 308, the computing device may determine whether the representation of the second catheter is detected within the imagery.

If the computing device detects the second catheter within the imagery (i.e., determination block 308=“Yes”), the computing device may determine whether the second catheter tip is at the desired location within the imagery in determination block 310. If the computing device determines that the second catheter tip is at the desired location within the imagery (i.e., determination block 310=“Yes”), the computing device may continue receiving additional ultrasound data for processing in block 302. If the computing device determines that the second catheter tip is not at the desired location within the imagery (i.e., determination block 310=“No”), the computing device may calculate positioning corrections based on the current location of the second catheter tip detected within the imagery in block 316 as described above with reference to FIG. 3.

If the computing device does not detect the second catheter within the imagery (i.e., determination block 308=“No”), in block 402 the computing device may calculate positioning correction's based on context information from the performed image analysis (e.g., heart geography, etc.), such as to search for the target catheter. As described above, the computing device may evaluate predefined organ model data (e.g., stored data indicating the dimensions or characteristics of a heart, etc.) and/or the ultrasound imagery to determine where with the ultrasound catheter is currently located within the patient's organ. For example, the computing device may determine that based on the lack of movement within the ultrasound imagery, the ultrasound catheter is rotated away from the portion of the heart that is the site for the procedure, and thus should be rotated a number of degrees in order to capture images of the second catheter. In an embodiment, the computing device may also evaluate predefined organ model data, such as mappings of the organ, individually or in combination with the context information in order to calculate the positioning correction information. As part of the operations of block 402, the computing device may calculate positioning correction that may be the most likely to position the ultrasound catheter in the direction of the second catheter, or as part of a predefined search pattern. Thus, the operations in block 402 may involve an automated procedure for iteratively searching for the second catheter using best guess repositioning.

After calculating positioning correction in block 402 or block 316, the computing device may generate repositioning instructions for a remotely-controlled catheter positioning system based on the calculated positioning correction (e.g., translate, rotate, bend, etc.) in block 318. The computing device may transmit the generated repositioning control instructions to a remotely-controlled catheter positioning system associated with the ultrasound catheter in block 320, and continue receiving and processing imagery in block 302 in a continuous process.

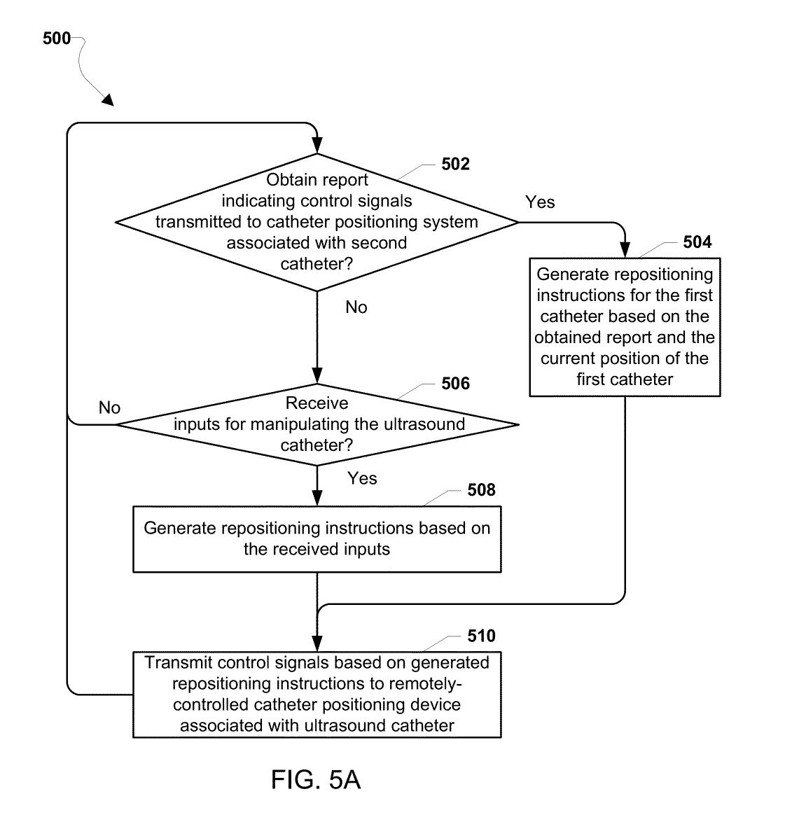

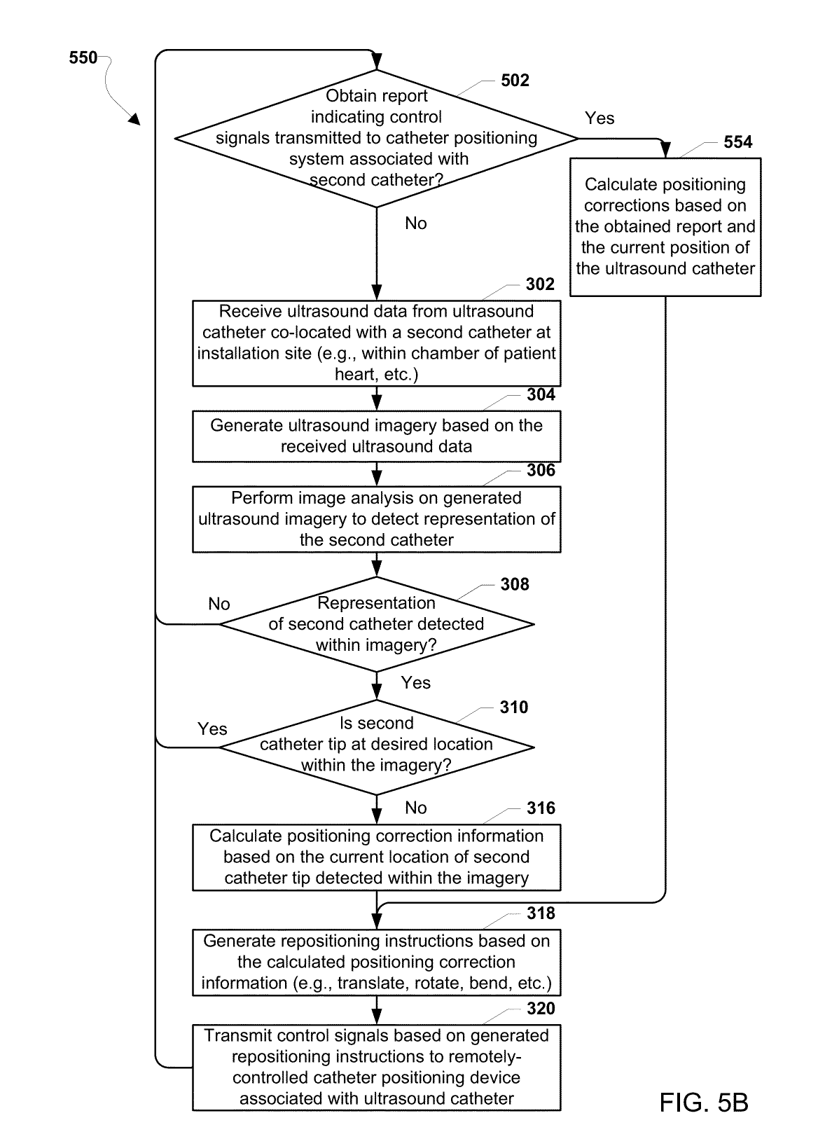

FIG. 5A illustrates an embodiment method 500 for a computing device to generate repositioning instructions for a remotely-controlled catheter positioning system to position a first catheter based on obtained control signals sent to a remotely-controlled catheter positioning system controlling a second catheter. In this embodiment method, the catheters may be moved in tandem without requiring ultrasound imagery analysis or additional inputs, such as from a second remote controller. The method 500 may be performed to automatically position any type of first catheter that is capable of being positioned with a remotely-controlled catheter positioning device, such as described below with reference to FIG. 9.

In determination block 502, the computing device may determine whether a report of repositioning control signals transmitted to a remotely-controlled catheter positioning system associated with the second catheter is obtained. Such a report may be received as a message from another device, such as a computer connected to the second remotely-controlled catheter positioning system. In another embodiment, the report may be data within a buffer or other data structure within the computing device. For example, the computing device may execute separate routines that are configured to process inputs associated with different remotely-controlled catheter positioning systems (and related catheters), and in response to receiving inputs for positioning the second catheter, the computing device may indicate the received inputs in a stored buffer that may be accessed by routines for automatically controlling another remotely-controlled catheter positioning system associated with the first catheter. If the computing device determines that a report indicating repositioning control signals transmitted to the remotely-controlled catheter positioning system associated with the second catheter was received (i.e., determination block 502=“Yes”), the computing device may generate repositioning instructions for the remotely-controlled catheter positioning system associated with the first catheter based on the received report and the current position of the first catheter in block 504. The computing device may determine the repositioning instructions to emulate the actions corresponding to the control signals within the report. For example, the computing device may generate a rotate instruction for the first catheter based on a control signal that was sent to cause the second catheter to move in a direction roughly parallel to the short axis of the ultrasound image.

Because the first and second catheters are not in the same location within the patient's organ, the computing device may also have to adjust instructions to avoid collisions with anatomy or other elements within the patient's organ. For example, a certain translation by the imaging catheter may not be possible when that translation would cause the imaging catheter to contact the wall of a heart chamber. The computing device may evaluate the current position of the first catheter and generate repositioning instructions that are appropriate for the difference in catheter positioning within the patient's organ and to maintain the first catheter within the field of view of the imaging catheter.

However, if the computing device determines that no report indicating repositioning control signals transmitted to the remotely-controlled catheter positioning system associated with the second catheter was received (i.e., determination block 502=“No”), the computing device may determine whether inputs for manipulating the first catheter are received, such as via a remote controller corresponding to the remotely-controlled catheter positioning system associated with the first catheter and connected to the computing device. If no inputs are received (i.e., determination block 506=“No”), the computing device may continue to monitor for receipt of report indicating repositioning control signals in determination block 502. If inputs for manipulating the first catheter are received (i.e., determination block 506=“Yes”), in block 508 the computing device may generate repositioning instructions based on the received inputs, such as bend, translate, and/or rotate instructions.

Based on the repositioning instructions generated in block 504 or block 508, the computing device may transmit repositioning control instructions to the remotely-controlled catheter positioning system associated with the first catheter in block 510. The computing device may then continue to monitor for receipt of report indicating repositioning control signals in determination block 502.

FIG. 5B illustrates an embodiment method 550 for a computing device to generate repositioning instructions for a remotely-controlled catheter positioning system to position an ultrasound catheter based on obtained control signals configured to position a second catheter. The method 550 includes operations similar to as described above with reference to the methods 300, 400, and 500, except that the method 550 also includes operations for generating repositioning instructions and related control signals for the ultrasound catheter in response to obtained reports of control signals associated with the second catheter. In other words, the computing device may automatically cause the ultrasound catheter to be repositioned within an organ based on contemporaneous movements of the second catheter and/or ultrasound imagery. For example, the computing device may cause the ultrasound catheter to be moved in a general manner the same amount and direction as the second catheter is moved based on user inputs via a remote controller, and then the ultrasound may be moved in a fine manner based on analysis of ultrasound imagery.

As described above, in determination block 502, the computing device may determine whether a report indicating repositioning control signals transmitted to a remotely-controlled catheter positioning system associated with the second catheter is obtained. If computing device determines that the report indicating repositioning control signals transmitted to the remotely-controlled catheter positioning system associated with the second catheter is obtained (i.e., determination block 502=“Yes”), the computing device may calculate positioning corrections based on the obtained report and the current position of the ultrasound catheter in block 554. The calculated positioning corrections may be an estimate of how much the ultrasound catheter should be moved to keep the second catheter in the center of the ultrasound imagery. For example, the computing device may calculate the distance the second catheter's tip likely moved based on the obtained report of the control signals that moved the second catheter.

If the computing device determines that no report indicating repositioning control signals transmitted to the remotely-controlled catheter positioning system associated with the second catheter has been obtained (i.e., determination block 502=“No”), the computing device may receive ultrasound data from an ultrasound catheter co-located with a second catheter within an organ in block 302, generate ultrasound imagery based on the received ultrasound data in block 304, and perform image analysis operations on the generated ultrasound imagery to detect a representation of the second catheter in block 306 as described above with reference to FIG. 3. In determination block 308, the computing device may determine whether the representation of the second catheter is detected within the imagery. If the computing device is not detect the second catheter within the imagery (i.e., determination block 308=“No”), the computing device may receive additional ultrasound data for processing in block 302.

If the computing device detects the second catheter within the imagery (i.e., determination block 308=“Yes”), the computing device may determine whether the second catheter tip is at the desired location within the imagery in determination block 310. If the computing device determines that the second catheter tip is at the desired location within the imagery (i.e., determination block 310=“Yes”), the computing device may continue to receive additional ultrasound data for processing in block 302.

If the computing device determines that the second catheter tip is not at the desired location within the imagery (i.e., determination block 310=“No”), the computing device may calculate positioning corrections based on the current location of the second catheter tip detected within the imagery in block 316. In block 318, the computing device may generate repositioning instructions for a remotely-controlled catheter positioning system based on the calculated positioning corrections (e.g., translate, rotate, bend, etc.). In block 320, the computing device may transmit the repositioning control instructions to a remotely-controlled catheter positioning system controlling with the ultrasound catheter. The computing device may perform the above-described operations in a continuous process by determining whether subsequent control signal reports are received in determination block 502.

Any type of computing device or combination of computing devices may be configured to perform some or all of the operations described above with reference to FIGS. 3-5B. For example, a server, a workstation, a dedicated system computer, or a desktop computer communicating with a data connection for exchanging data with an ultrasound imaging system may be configured to use the ultrasound imagery to generate repositioning instructions for a remotely-controlled catheter positioning device.

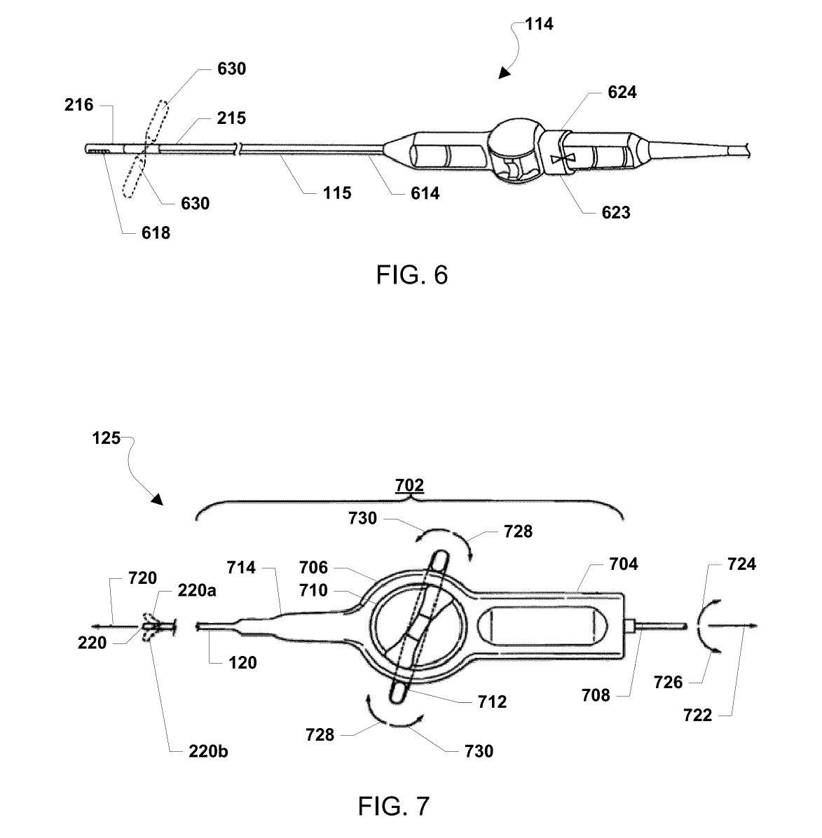

The embodiments may be implemented with a variety of different types of catheter devices that are well known in the art, such as ablation catheters and electrophysiology catheters. Therefore, the devices described below with reference to FIGS. 6-7 should be considered examples of devices that may be used with the various embodiments that are provided for illustration purposes only.

FIG. 6 illustrates an example ultrasound catheter 114 suitable for use in various embodiments. As is well-known in the art, the ultrasound catheter 114 may be configured to perform ultrasound imaging and to be steerable through various patient's organs, such as human anatomy (e.g., cardiovascular systems, etc.). The ultrasound catheter 114 may include an elongated tubular member 115 that may be made of various materials, such as extruded polyether block amide, polyethylene, silicone rubber, plasticized PVC, and/or polymeric materials. The ultrasound catheter 114 may be configured with different sections of varying flexibility so that the tubular member 115 may bend and otherwise be contorted within a patient's organ. The ultrasound catheter 114 may be of various lengths appropriate for use in various intravascular procedures. For example, the ultrasound catheter 114 may be 80 cm in insertable length, 90 cm in insertable length, 120 cm in insertable length, etc.

A tubular member 115 of the ultrasound catheter 114 may have a proximal end 614 (or proximal portion) and a distal end 215 (or distal portion). Distal portions of the tubular member 115 may be more flexible than proximal portions to improve maneuverability and decrease the risk of damage to a patient's organ. Like other known catheters, the ultrasound catheter 114 may also include a steering mechanism 624 that may control tensions on steering cables (not shown) within the tubular member 115 and may cause bending or other contorting of the tubular member 115. The steering mechanism may be a handle, a slide actuator, a rotatable control knob, handle or wheel, or other suitable manipulating member mounted in a control handle 623.

An ultrasound transducer 216 may be at the distal end 215 of the tubular member 115. The transducer 216 may be formed from an array of individual ultrasound elements 618. As is well known in the art, the transducer 216 may be comprised of various numbers and configurations of ultrasound elements 618, such as forty-eight elements or sixty-four elements to form a linear phased array ultrasound imaging sensor. The transducer 216 may be connected to a plurality of electrical cables (e.g., coaxial cables) running through the tubular member 115. There may be one electrical cable per each ultrasound element 618 of the transducer 216. In order to fit various components, such as the transducer 216 and associated wires, the diameter of the ultrasound catheter 114 may be in various size ranges, such as in between 6 to 12 French. The direction or orientation of the transducer 216 and the ultrasound elements 618 may be adjusted by bending the tubular member 115 of the catheter 114, such as by manipulating (or steering) the ultrasound catheter 114 via a remote control or device. Such bending of the ultrasound catheter 114 is shown with the dotted lines 630 in FIG. 6. Different ultrasound imaging angles may be achieved through manipulating the shape (e.g., bending) or position of the catheter 114. The ultrasound catheter 114 may include various conduits and sheaths (not shown) for protecting the elements within the tubular member 115.

FIG. 7 illustrates another example of a catheter 125 that may be used in accordance with various embodiments. The catheter 125 may include a catheter handle 702 which may be gripped by a clinician. The catheter handle 702 may include a proximal end 704 and a grip 706. Inserted into the proximal end 704 may be wires 708 or tubing which could provide electricity, coolant, heat, etc., to the catheter 125. The grip 706 may include an adjustment dial 710 which may be used to adjust the tension of a knob 712. The catheter handle 702 may terminate in a distal flexible end portion 714 which in turn may be in communication with a distally extending catheter sheath or tubular member 120.

As it is known in the art, a tubular member 120 (e.g., a catheter sheath) may be inserted into a patient by use of various known procedures and devices. The tubular member 120 may terminate in a distal end 220. The distal end 220 may include, for example, electrodes for supplying electrical stimulation, coolant, heat, etc.

The tubular member 120 may be physically attached to the catheter handle 702 so that movement of the catheter handle 702 forward or backward in the direction of arrow 720 or 722 may cause the tubular member 120, as well as the distal end 220, to move similarly. Rotation or torquing of the catheter handle 702 in a clockwise or counterclockwise manner as is shown by arrows 724 and 726, may impart a similar rotation to the tubular member 120. Rotation of the knob 712 in the direction of arrow 728 or 730 may cause deflection of the distal end 220 in one of the directions shown as 220a and 220b. Thus, when used manually, commercially available catheters may operate in six ranges of motion: forward and backward in the direction of arrows 720 and 722, rotation in the direction of arrows 724 and 726, and deflection to positions such as 220a and 220b.

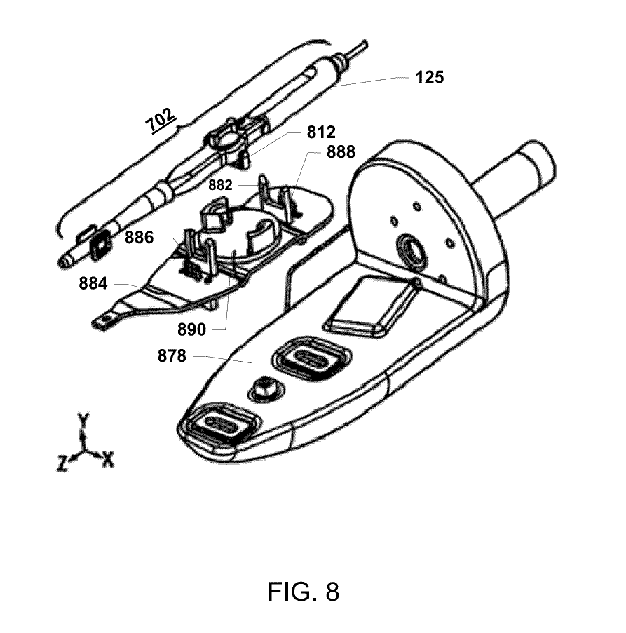

FIG. 8 illustrates some example components used by a remotely-controlled catheter positioning system suitable for use in various embodiments. A sled member 878 may be capable of receiving a catheter control handle in a handle control assembly 882 and mounted to a modular plate 884. A handle control assembly 882 may include clamps 886 and 888 and a molded nest 890. The knob 812 of the catheter handle 702 may be secured in the molded nest 890 by friction or snap-in fit.

The sled member 878 may be attached to the catheter handle 702 by the modular plate 884 and the handle control assembly 882. The modular plate 884 and handle control assembly 882 may be specific to the type/manufacture of the catheter 125 shown. Different modular plates 884 and handle control assemblies 882 may be used dependent upon the type/make of catheter used. The modular plates 884 and handle control assemblies 882 may be sterilizable, disposable, or both. As the modular plate 884 is detachable from sled member 878, different handles may be used for different types of catheters.

It should be noted that various types of available, off-the-shelf or other catheter may be utilized in remotely-controlled catheter positioning systems, such as a bi-directional ablation catheter or the ultrasound catheter 114 described above with reference to FIG. 6. For example, a cardiac ablation catheter with a corresponding modular plate 884 may be used. A fastening mechanism may include clamps, such as clamps 886 and 888 may attach the catheter 125 to the modular plate 884.

The catheter handle 702 of catheter 125 may be engaged into the modular plate 884 at three points, namely, clamps 886 and 888 and the molded nest 890. The catheter handle 702 may be snap fit into molded nest 890 or secured by friction. The proximal end of catheter handle 702 may be mounted to the modular plate 884 through the use of the clamp 886 and the distal end may be mounted onto the modular plate 884 through the use of the clamp 888. The clamps 886, 888 may be snap fit. If the catheter 125 has an additional range of motion, such as the point of deflection in a certain ablation catheter, an additional motor may be attached to move the corresponding control on the handle. The modular plate 884 may subsequently be attached to the sled member 878 by snap fit. The modular plate 884 may have protrusions (not shown) effective to secure the modular plate 884 to the sled member 878. The modular plate 884 may also be attached to a sled member and the catheter handle.

The sled member 878 may be equipped with rear and/or front end force sensors (not shown) to gauge force in three zones. A display (not shown) may be located on modular plate 884 or elsewhere. The display may indicate forces of low, medium, and high. These indications may be represented by colored lights, including green, yellow, and red respectively, or bars of light, such as one bar, two bar, or three bars. In a further embodiment, the display may further include an audio sensor which emits a noise when the incorrect amount of force is applied.

FIG. 9 illustrates an example remotely-controlled catheter positioning system 110 and remote controller 102 suitable for use in various embodiments. The remotely-controlled catheter positioning system 110 may include a linear sled bed or sled base 936 which supports a linear sled member 938, a mounting arm 940 which supports sled base 936, a sterile guide barrier 942, a handle controller 944, a catheter dock or handle control assembly 946, a catheter introducer 943, and a catheter introducer coupling 950. In an embodiment, the sled base 936 may be positioned using a local control and positioning handle 952 or a remote controller 954. The mounting arm 940 may connect to the sled base 936 and allow for vertical (downward and upward) rotational motion and horizontal (left and right) rotational motion. The mounting arm 940 may be moved manually or mechanically through the use of a remote controller 102. The mounting arm 940 may be attached to either the left or right side bars of an operative surface 956, such as a fluoroscopy table, and may optionally be further attached to the foot of the table with a third support in a tripod-like configuration. Alternatively, a circular monorail or other configuration of rails may support one or more devices (e.g., robotic devices) for the purpose of remote mapping and ablation or one or more catheters.

A motor housing 958 may house a motor (not shown) mounted on a support surface (not shown). Such a motor may receive power and signal control through the use of wires fed through a wire housing (not shown) and terminal connectors (not shown). Wires may supply both power and signal control to the motor and the handle controller 944. The motor may rotate a drive screw (not shown) to advance the sled member 938. The motor may easily move the handle controller 944 and sled member 938 back and forth on the sled base 936 to help with catheter placement. The handle controller 944 may be coupled to the sled member 938. The device 110 may be connected to a control device or system (not shown), such as a programmable computing device, via a connection 111.

The remotely-controlled catheter positioning system 110 may be connected to the remote controller 102 which may be used to receive inputs from clinicians (or users). The remote controller 102 may imitate the look and feel of a standard catheter for ease in controls, and further may be designed for use with a single hand. The distal end 972 may be rotated to control right roll and left roll of a catheter, such as catheter 125 described above. Buttons (not shown) located on the body of the remote controller 102 may control in and out functionalities. At the indentation close to the distal end 972 of remote controller 102, a knob 974 may be used to control deflection or other articulation. A connection 103 (or wire) located at proximal end 976 may connect the remote controller 102 to a power source. In various configurations, the remote controller 102 may be connected to the remotely-controlled catheter positioning system 110 and/or a control device or system (e.g., a computing device) via the connection 103.

Various other catheters, medical devices, and configurations and components for remotely-controlled catheter positioning systems and related systems that may be used with the embodiments are described in U.S. patent application Ser. No. 13/432,161 that is incorporated by reference above.

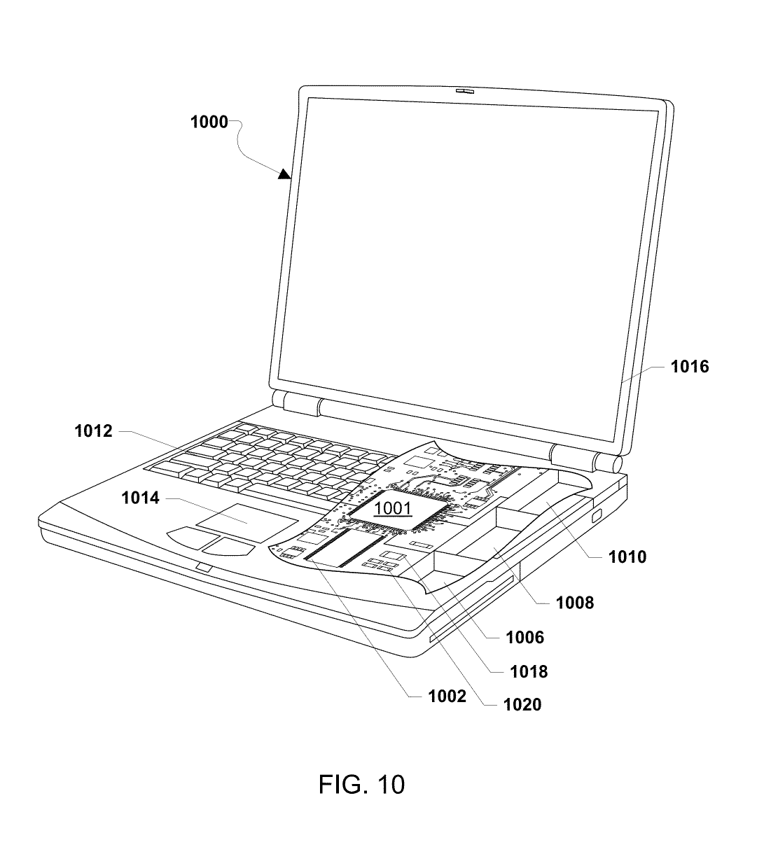

A number of different types of computing devices may be used to implement the various embodiments, including personal computers and laptop computers. Such computing devices typically include the components illustrated in FIG. 10 which illustrates an example laptop computing device 1000. Many laptop computing devices 1000 may include a touch pad touch surface 1014 that serves as the computer's pointing device, and thus may receive drag, scroll, and flick gestures similar to those implemented on mobile computing devices equipped with a touch screen display and described above. Such a laptop computing device 1000 may generally include a processor 1001 coupled to volatile internal memory 1002 and a large capacity nonvolatile memory, such as a disk drive 1006. The laptop computing device 1000 may also include a compact disc (CD) and/or DVD drive 1008 coupled to the processor 1001. The laptop computing device 1000 may also include a number of connector ports 1010 coupled to the processor 1001 for establishing data connections or receiving external memory devices, such as a network connection circuit for coupling the processor 1001 to a network. The laptop computing device 1000 may have one or more short-range radio signal transceivers 1018 (e.g., Peanut®, Bluetooth®, Zigbee®, RF radio) and antennas 1020 for sending and receiving wireless signals as described herein. The transceivers 1018 and antennas 1020 may be used with the above-mentioned circuitry to implement the various wireless transmission protocol stacks/interfaces. In a laptop or notebook configuration, the computer housing may include the touch pad 1014, the keyboard 1012, and the display 1016 all coupled to the processor 1001. Other configurations of the computing device may include a computer mouse or trackball coupled to the processor (e.g., via a USB input) as are well known, which may also be used in conjunction with the various embodiments.

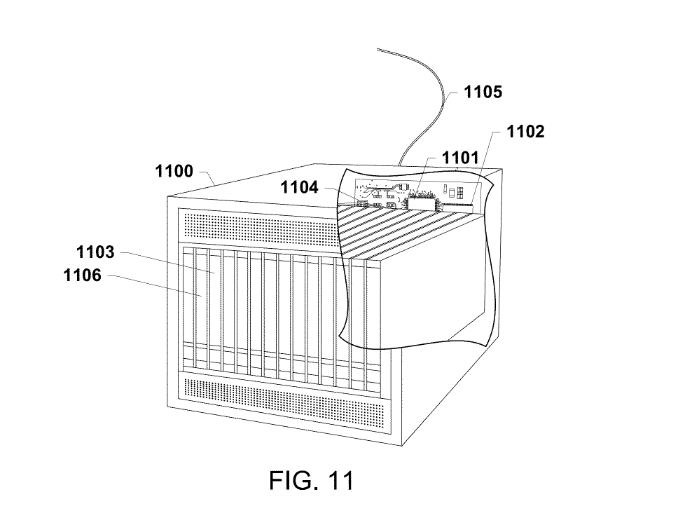

The various embodiments may be implemented on any of a variety of commercially available computing devices, such as the workstation computer 1100 illustrated in FIG. 11. Such a workstation computer 1100 may typically include a processor 1101 coupled to volatile memory 1102 and a large capacity nonvolatile memory, such as a disk drive 1103. The workstation computer 1100 may also include a floppy disc drive, compact disc (CD) or DVD disc drive 1106 coupled to the processor 1101. The workstation computer 1100 may also include network access ports 1104 coupled to the processor 1101 for establishing data connections with a network 1105 or other devices, such as a local area network coupled to other medical devices and servers.

The processors 1001 and 1101 may be any programmable microprocessor, microcomputer or multiple processor chip or chips that can be configured by software instructions (applications) to perform a variety of functions, including the functions of the various embodiments described above. In the various devices, multiple processors may be provided, such as one processor dedicated to wireless communication functions and one processor dedicated to running other applications. Typically, software applications may be stored in the internal memory 1002 and 1102 before they are accessed and loaded into the processors 1001 and 1101. The processors 1001 and 1101 may include internal memory sufficient to store the application software instructions. In many devices the internal memory may be a volatile or nonvolatile memory, such as flash memory, or a mixture of both. For the purposes of this description, a general reference to memory refers to memory accessible by the processors 1001 and 1101 including internal memory or removable memory plugged into the various devices and memory within the processors 1001 and 1101.

The foregoing method descriptions and the process flow diagrams are provided merely as illustrative examples and are not intended to require or imply that the steps of the various embodiments must be performed in the order presented. As will be appreciated by one of skill in the art the order of steps in the foregoing embodiments may be performed in any order. Words such as “thereafter,” “then,” “next,” etc. are not intended to limit the order of the steps; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The hardware used to implement the various illustrative logics, logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some steps or methods may be performed by circuitry that is specific to a given function.

In the functions of the various embodiments described above may be implemented in hardware, software, firmware, or any combination thereof. If implemented in software, the functions may be stored on or transmitted over as one or more instructions or code on a non-transitory processor-readable (i.e., processor-readable instructions), computer-readable medium or a non-transitory processor-readable storage medium. The steps of a method or algorithm disclosed herein may be embodied in a processor-executable software module or processor-executable software instructions which may reside on a non-transitory computer-readable storage medium, and/or a non-transitory processor-readable storage medium. In various embodiments, such instructions may be stored processor-executable instructions or stored processor-executable software instructions. Tangible, non-transitory computer-readable storage media may be any available media that may be accessed by a computer. By way of example, and not limitation, such non-transitory computer-readable media may comprise RAM, ROM, EEPROM, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and blu-ray disc where disks usually reproduce data magnetically, while discs reproduce data optically with lasers. Combinations of the above should also be included within the scope of non-transitory computer-readable media. Additionally, the operations of a method or algorithm may reside as one or any combination or set of codes and/or instructions on a tangible, non-transitory processor-readable storage medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

What is claimed is:

1. A method for a computing device to generate repositioning instructions for a first remotely-controlled catheter positioning system to position a first catheter that is co-located with a second catheter within a patient, comprising:
performing image analysis on imagery generated based on data from the first catheter;
calculating positioning corrections based on the performed image analysis;
generating repositioning instructions based on the calculated positioning corrections; and
transmitting the generated repositioning instructions to the first remotely-controlled catheter positioning system.

2. The method of claim 1, further comprising determining whether the second catheter appears within the imagery based on the performed image analysis, and
wherein calculating positioning corrections based on the performed image analysis comprises calculating the positioning corrections in response to determining that the second catheter appears within the imagery based on the performed image analysis.

3. The method of claim 1, further comprising determining whether a tip of the second catheter is at a desired location within the imagery based on the performed image analysis, and
wherein calculating positioning corrections based on the performed image analysis comprises calculating the positioning corrections based on a current location of the tip of the second catheter within the imagery in response to determining that the tip of the second catheter is within the imagery but not at the desired location within the imagery.

4. The method of claim 1, further comprising:
rendering a prompt requesting user input to confirm automated repositioning operations; and
determining whether a user input is received confirming automated repositioning operations,
wherein transmitting control signals based on the generated repositioning instructions to the first remotely-controlled catheter positioning system comprises transmitting the control signals based on the generated repositioning instructions to the first remotely-controlled catheter positioning system in response to determining that a user input confirming automated repositioning operations is received.

5. The method of claim 1, further comprising calculating the positioning corrections using context information from the performed image analysis.

6. The method of claim 1, further comprising:
obtaining a report indicating control signals transmitted to a second remotely-controlled catheter positioning system for repositioning the second catheter; and
generating the repositioning instructions for the first remotely-controlled catheter positioning system based on the obtained report.

7. The method of claim 1, wherein the first catheter is an ultrasound catheter.

8. A computing device configured for controlling a first remotely-controlled catheter positioning system, comprising a processor configured with processor-executable instructions to cause the computing device to perform operations comprising:
performing image analysis on imagery generated based on data from a first catheter;
calculating positioning corrections based on the performed image analysis;
generating repositioning instructions based on the calculated positioning corrections; and
transmitting the generated repositioning instructions to the first remotely-controlled catheter positioning system.

9. The computing device of claim 8, wherein the processor is configured with processor-executable instructions to cause the computing device to perform operations further comprising determining whether a second catheter appears within the imagery based on the performed image analysis, and
wherein the processor is configured with processor-executable instructions to cause the computing device to perform operations such that calculating positioning corrections based on the performed image analysis comprises calculating the positioning corrections in response to determining that the second catheter appears within the imagery based on the performed image analysis.

10. The computing device of claim 8, wherein the processor configured with processor-executable instructions to cause the computing device to perform operations further comprising determining whether a tip of a second catheter is at a desired location within the imagery based on the performed image analysis, and
wherein the processor is configured with processor-executable instructions to cause the computing device to perform operations such that calculating positioning corrections based on the performed image analysis comprises calculating the positioning corrections based on a current location of the tip of the second catheter within the imagery in response to determining that the tip of the second catheter is within the imagery but not at the desired location within the imagery.

11. The computing device of claim 8, wherein the processor is configured with processor-executable instructions to cause the computing device to perform operations further comprising:
rendering a prompt requesting user input to confirm automated repositioning operations; and
determining whether a user input is received confirming automated repositioning operations,
wherein the processor is configured with processor-executable instructions to cause the computing device to perform operations such that transmitting control signals based on the generated repositioning instructions to the first remotely-controlled catheter positioning system comprises transmitting the control signals based on the generated repositioning instructions to the first remotely-controlled catheter positioning system in response to determining that a user input confirming automated repositioning operations is received.

12. The computing device of claim 8, wherein the processor is configured with processor-executable instructions to cause the computing device to perform operations further comprising calculating the positioning corrections using context information from the performed image analysis.

13. The computing device of claim 8, wherein the processor is configured with processor-executable instructions to cause the computing device to perform operations further comprising:
obtaining a report indicating control signals transmitted to a second remotely-controlled catheter positioning system associated with a second catheter; and
generating the repositioning instructions for the first catheter based on the obtained report.

14. A system comprising:
a first remotely-controlled catheter positioning system comprising:
a modular plate configured to receive a proximal portion of a catheter;
a sled member coupled to the modular plate; and
a sled base configured to advance the sled member along a guide towards a body of a patient;
an imaging catheter coupled to the first remotely-controlled catheter positioning system; and
a computing device comprising a processor configured with executable instructions to cause the computing device to perform operations comprising:
performing image analysis on imagery generated based on data collected from the imaging catheter;
calculating positioning corrections based on the performed image analysis;
generating repositioning instructions based on the calculated positioning corrections configured to control the first remote-controlled catheter positioning system in order to maintain an object within a field of view of the imaging catheter; and
transmitting control signals based on the generated repositioning instructions to the first remotely-controlled catheter positioning system.

15. The system of claim 14, wherein the processor is configured with executable instructions to cause the computing device to perform operations further comprising determining whether a second catheter appears within the imagery based on the performed image analysis, and
wherein the processor is configured with executable instructions to cause the computing device to perform operations such that calculating positioning corrections based on the performed image analysis comprises calculating the positioning corrections in response to determining that the second catheter appears within the imagery based on the performed image analysis.

16. The system of claim 14, wherein the processor is configured with executable instructions to cause the computing device to perform operations further comprising determining whether a tip of a second catheter is at a desired location within the imagery based on the performed image analysis, and
wherein the processor is configured with executable instructions to cause the computing device to perform operations such that calculating positioning corrections based on the performed image analysis comprises calculating the positioning corrections based on a current location of the tip of the second catheter within the imagery in response to determining that the tip of the second catheter is within the imagery but not at the desired location within the imagery.

17. The system of claim 14, wherein the processor is configured with executable instructions to cause the computing device to perform operations further comprising:
rendering a prompt requesting user input to confirm automated repositioning operations; and
determining whether a user input is received confirming automated repositioning operations,
wherein the processor is configured with executable instructions to cause the computing device to perform operations such that transmitting control signals based on the generated repositioning instructions to the first remotely-controlled catheter positioning system comprises transmitting the control signals based on the generated repositioning instructions to the first remotely-controlled catheter positioning system in response to determining that a user input confirming automated repositioning operations is received.

18. The system of claim 14, wherein the processor is configured with executable instructions to cause the computing device to perform operations further comprising calculating the positioning corrections using context information from the performed image analysis.

19. The system of claim 14, further comprising a second remotely-controlled catheter positioning system coupled to second catheter, wherein the processor is configured with executable instructions to cause the computing device to perform operations further comprising:
obtaining a report indicating control signals transmitted to the second remotely-controlled catheter positioning system for repositioning the second catheter; and
generating the repositioning instructions for the first remotely-controlled catheter positioning system based on the obtained report.
